(19) United States
(12) Patent Application Publication
(10) Pub. No.: US 2023/0013824 A1
(43) Pub. Date: Jan. 19, 2023

(54) COMPOSITIONS FOR THE TREATMENT OF HYPERKERATOSIS DISORDERS

(71) Applicant: Azura Ophthalmics Ltd., Tel Aviv (IL)

(72) Inventors: Yair ALSTER, Tel Aviv (IL); Omer RAFAELI, Udim (IL); Doron FRIEDMAN, Carme-yosrf (IL); Zakhar NUDELMAN, Netanya (IL); Shimon AMSELEM, Rehovot (IL)

(21) Appl. No.: 17/725,320

(22) Filed: Apr. 20, 2022

Related U.S. Application Data
(63) Continuation of application No. 16/093,616, filed on Oct. 12, 2018, now abandoned.
(60) Provisional application No. 62/324,795, filed on Apr. 19, 2016.

(51) Int. Cl. A61K 31/095; A61P 17/12; A61K 9/00; A61K 31/198; A61K 33/04

(52) U.S. Cl. CPC A61K 31/095; A61P 17/12; A61K 9/0014; A61K 9/0048; A61K 31/198; A61K 33/04; A61K 9/0019; A61K 9/08

(57) ABSTRACT

Described herein are compositions and methods for treating hyperkeratosis disorders such as dandruff, psoriasis, acne vulgaris, warts, corns, calluses, palmoplantar keratodermas, ichthyosis, seborrheic dermatitis, meibomian gland dysfunction, HPV infection, lichen planus, aclinic keratosis, seborrheic keratosis, etc. Said compositions comprise a selenium-containing amino acid as keratolytic agent, and are topically administered to the skin or eyelid margin of the patient. In some embodiments, the composition comprises a selenium-containing amino acid such as selenium methionine or selenium cysteine formulated in a ophthalmic, dermatological, or cosmetic dosage form.

### CROSS REFERENCE

This application is a continuation of U.S. patent application Ser. No. 16/093,616, filed on Oct. 12, 2018, which is a U.S. National Stage Entry of International Application No. PCT/IB2017/000638, filed on Apr. 18, 2017, which claims the benefit of U.S. Provisional Application No. 62/324,795, filed on Apr. 19, 2016, all of which are incorporated by reference herein in their entireties.

### BACKGROUND OF THE INVENTION

Hyperkeratosis disorders are characterized by the thickening of the stratum corneum (the outermost layer of the epidermis), are often associated with the presence of an abnormal quantity of keratin, and are also usually accompanied by an increase in the granular layer. The elevated levels of keratin contribute to hyperkeratosis disorders such as dandruff, psoriasis, acne vulgaris, warts, corns, calluses, palmoplantar keratodermas, ichtiosis, seborrheic dermatitis, meibomian gland dysfunction, HPV infection, lichen planus, actinic keratosis, and seborrheic keratosis. New therapeutic approaches to the treatment of hyperkeratosis disorders are needed.

### SUMMARY OF THE INVENTION

One embodiment provides a composition for treating hyperkeratosis disorders in a patient in need thereof comprising a selenium-containing amino acid as a keratolytic agent and wherein the composition is suitable for topical administration to the skin in a pharmaceutical or cosmetic dosage form. Another embodiment provides the composition wherein the selenium-containing amino acid is selenium methionine, or a pharmaceutically acceptable salt thereof. Another embodiment provides the composition wherein the selenium-containing amino acid is selenium cysteine, or a pharmaceutically acceptable salt thereof.

Another embodiment provides the composition wherein the composition comprises the selenium-containing amino acid in a liquid solution formulation for ophthalmic, dermatological, or cosmetic use. Another embodiment provides the composition wherein the composition comprises the selenium-containing amino acid in a suspension for ophthalmic, dermatological, or cosmetic use. Another embodiment provides the composition wherein the composition comprises the selenium-containing amino acid in a liquid gel for ophthalmic, dermatological, or cosmetic use.

Another embodiment provides the composition wherein the composition is a cream for ophthalmic, dermatological, or cosmetic use. Another embodiment provides the composition wherein the composition is an emulsion for ophthalmic, dermatological, or cosmetic use. Another embodiment provides the composition wherein the composition is a lotion for ophthalmic, dermatological, or cosmetic use. Another embodiment provides the composition wherein the composition is an ointment for ophthalmic, dermatological, or cosmetic use.

One embodiment provides a composition for treating hyperkeratosis disorders in a patient in need thereof comprising a selenium-containing amino acid as a keratolytic agent and wherein the composition is suitable for topical administration to the skin in a pharmaceutical or cosmetic dosage form, wherein the composition further comprises an additional keratolytic or keratostatic agent selected from benzoyl peroxide, coal tar, dithranol, salicylic acid, retinoic acid, alpha-hydroxy acid, urea, lactic acid and selenium disulfide.

One embodiment provides a composition for treating hyperkeratosis disorders in a patient in need thereof comprising a selenium-containing amino acid as a keratolytic agent and wherein the composition is suitable for topical administration to the skin in a pharmaceutical dosage form, wherein the composition is administered via a depot formulation and the depot further comprises a pressure sensitive adhesive, wherein the pressure sensitive adhesive is a rubber based pressure sensitive adhesive, a silicone based pressure sensitive adhesive, or an acrylic based pressure sensitive adhesive.

One embodiment provides a method for treating a hyperkeratosis disorder in a patient in need thereof comprising topically administering to the skin or eyelid margin of the patient a composition comprising selenium methionine or selenium cysteine formulated as an ophthalmic, dermatological, or cosmetic dosage form.

Another embodiment provides the method wherein the composition is formulated as a solution for ophthalmic, dermatological, or cosmetic use. Another embodiment provides the method wherein the composition is formulated as a suspension for ophthalmic, dermatological, or cosmetic use. Another embodiment provides the method wherein the composition is formulated as a lotion for ophthalmic, dermatological, or cosmetic use. Another embodiment provides the method wherein the composition is formulated as a cream for ophthalmic, dermatological, or cosmetic use. Another embodiment provides the method wherein the composition is formulated as an ointment for ophthalmic, dermatological, or cosmetic use. Another embodiment provides the method wherein the composition is formulated as a gel for ophthalmic, dermatological, or cosmetic use. Another embodiment provides the method wherein the composition is formulated as an emulsion for ophthalmic, dermatological, or cosmetic use. Another embodiment provides the method wherein the composition is formulated as a semi-solid for ophthalmic, dermatological, or cosmetic use.

One embodiment provides a method for treating a hyperkeratosis disorder in a patient in need thereof comprising topically administering to the skin or eyelid margin of the patient a composition comprising selenium methionine or selenium cysteine formulated as an ophthalmic, dermatological, or cosmetic dosage form, wherein the composition for topical administration further comprises an additional keratolytic or keratostatic agent selected from benzoyl peroxide, coal tar, dithranol, salicylic acid, retinoic acid, alpha-hydroxy acid, urea, lactic acid and selenium disulfide.

One embodiment provides a method for treating a hyperkeratosis disorder in a patient in need thereof comprising topically administering to the skin or eyelid margin of the patient a composition comprising selenium methionine or selenium cysteine formulated as an ophthalmic, dermatological, or cosmetic dosage form, wherein the method comprises administration from a depot formulation and the depot formulation further comprises a pressure sensitive adhesive, wherein the pressure sensitive adhesive is a rubber based pressure sensitive adhesive, a silicone based pressure sensitive adhesive, or an acrylic based pressure sensitive adhesive.

One embodiment provides a method for treating a hyperkeratosis disorder in a patient in need thereof comprising topically administering to the skin or eyelid margin of the patient a composition comprising selenium methionine or selenium cysteine formulated as an ophthalmic, dermatological, or cosmetic dosage form, wherein the hyperkeratosis disorder is selected from dandruff, psoriasis, acne vulgaris, warts, corns, calluses, palmoplantar keratodermas, ichthyosis, seborrheic dermatitis, meibomian gland dysfunction, HPV infection, lichen planus, actinic keratosis, and seborrheic keratosis. Another embodiment provides the method wherein the hyperkeratosis disorder is meibomian gland dysfunction. Another embodiment provides the method wherein the hyperkeratosis disorder is dry eye.

Provided herein are compositions for treating hyperkeratosis disorders such as dandruff, psoriasis, acne vulgaris, warts, corns, calluses, palmoplantar keratodermas, ichthyosis, seborrheic dermatitis, meibomian gland dysfunction, HPV infection, lichen planus, actinic keratosis, seborrheic keratosis, etc, comprising a selenium-containing amino acid as a keratolytic agent, wherein the composition is suitable for topical administration to the hyperkeratotic area in a pharmaceutical or cosmetic dosage form. In some embodiments the selenium-containing amino acid is selenium methionine. In some embodiments the selenium-containing amino acid is selenium cysteine.

The present disclosure further provides compositions for treating hyperkeratosis disorders, wherein the composition for topical administration further comprises an additional keratolytic or keratostatic agent with additive or synergistic pharmacological effect selected from benzoyl peroxide, coal tar, dithranol, salicylic acid, retinoic acid, alpha-hydroxy acid, urea, lactic acid and selenium disulfide.

The present disclosure further provides methods for treating hyperkeratosis disorders such as dandruff, psoriasis, acne vulgaris, warts, corns, calluses, palmoplantar keratodermas, iichthyosis, seborrheic dermatitis, meibomian gland dysfunction, HPV infection, lichen planus, actinic keratosis, seborrheic keratosis, etc comprising topically administering to the skin or eyelid margin of the patient a composition comprising a selenium-containing amino acid formulated as ophthalmic, dermatological, or cosmetic dosage form. In some embodiments the selenium-containing amino acid is selenium methionine. In some embodiments the selenium-containing amino acid is selenium cysteine.

The present disclosure further provides methods for treating hyperkeratosis disorders, wherein the composition for topical administration further comprises an additional keratolytic or keratostatic agent with additive or synergistic pharmacological effect selected from benzoyl peroxide, coal tar, dithranol, salicylic acid, retinoic acid, alpha-hydroxy acid, urea, lactic acid and selenium disulfide.

### BRIEF DESCRIPTION OF THE DRAWINGS

FIG. 1 illustrates the dose-response and time course analysis of selenium methionine (FIG. 1A) and salicylic acid (SA) (FIG. 1B) induced reduction of keratinocytes proliferation in HaCaT cells (viability). HaCaT cells were incubated without or with increasing concentrations of selenium methionine or salicylic acid for 24, 48 and 72 hr. Cell viability was measured by MTT assay. Results are shown as percent of control. SDS (0.1%) served as positive control. Mean±SEM; n=3. *p<0.05 for reduction from untreated control.

FIG. 2 illustrates the dose-response of selenium methionine (FIG. 2A) and SA (FIG. 2B) induced apoptosis in HaCaT keratinocyte cells. HaCaT cells were incubated without or with increasing concentrations of selenium methionine for 24, 48 and 72 hr. Apoptosis was measured by the caspase-3 assay. Results are shown as percent of control. Staurosporine (100 nM) served as positive control. Mean±SEM; n=3. *p<0.05 for reduction from untreated control.

FIG. 3 illustrates the keratostatic effect of selenium methionine (FIG. 3A) and SA (FIG. 3B) in HaCaT keratinocyte cells measured using the BrdU assay. HaCaT cells were incubated without or with the indicated concentrations of selenium methionine for 48 hr. Turnover rate was measured by BrdU. Results are shown as percent of control. Staurosporine served as positive control. Mean±SEM; n=3. *p<0.05 for reduction from untreated control.

FIG. 4 illustrates the effect of selenium methionine (FIG. 4A) and SA (FIG. 4B) on cell cycle by FACS analysis. HaCaT keratinocyte cells were synchronized by serum deprivation for 24 hr. Then the cells were incubated without or with the indicated concentrations of selenium methionine or salicylic acid for 24 hr. Cell cycle was measured by FACS analysis. Results are shown as percent of control. Staurosporine (100 nM) served as positive control. Mean±SEM; n=3. *p<0.05 for reduction from untreated control.

FIG. 5 illustrates the keratolytic effect of selenium methionine (FIG. 5A) and SA (FIG. 5B) ex vivo in human skin tissue as determined by the thiol degradation assay. Human stratum corneum samples were incubated without or with the indicated concentrations of selenium methionine or salicylic acid for 2 hr. Then, the mixtures were analyzed for free thiol moieties concentration. Results are shown as percent of control. Mean±SEM;n=2. *p<0.05 for reduction from untreated control.

FIG. 6A illustrates the dose-response and time course analysis of selenium cysteine induced reduction of keratinocyte proliferation in HaCaT cells (viability). HaCaT cells were incubated without or with increasing concentrations of selenium cysteine for 24, 48 and 72 hr. Cell viability was measured by MTT assay (FIG. 6A). FIG. 6B illustrates the positive and negative control (SDS). As expected, SDS significantly decreased cell viability. Results are shown as percent of control. Mean±SEM; n=3. *p<0.05 for reduction from untreated control.

FIG. 7 illustrates the keratostatic effect of selenium cysteine in HaCaT keratinocyte cells measured using the BrdU assay. HaCaT cells were incubated without or with the indicated concentrations of selenium cysteine for 48 hr. Turnover rate was measured by BrdU. Results are shown as percent of control. Mean±SEM; n=3. *p<0.05 for reduction from untreated control.

### DETAILED DESCRIPTION OF THE INVENTION

The present disclosure provides compositions useful for treating hyperkeratosis disorders such as dandruff, psoriasis, acne vulgaris, warts, corns, calluses, palmoplantar keratodermas, ichthyosis, seborrheic dermatitis, meibomian gland dysfunction, HPV infection, lichen planus, actinic keratosis, seborrheic keratosis, etc, comprising a selenium-containing amino acid as a keratolytic agent. Further, the present disclosure provides methods for treating said hyperkeratosis disorders comprising administering a selenium-containing amino acid as a keratolytic agent and wherein the composition is suitable for topical administration to the skin in a pharmaceutical or cosmetic dosage form.

### Hyperkeratosis Dermal Conditions

Acne vulgaris is the most common skin disease. It is characterized by hyperkeratosis of the follicular epithelium, leading to horny impactions that may lie dormant as open or closed comedones or may cause inflammation of the follicle.

Seborrheic dermatitis also known as seborrhea, sebopsoriasis, seborrheic eczema, dandruff and pityriasis capitis is a chronic, relapsing and usually mild dermatitis. Seborrheic dermatitis is an inflammatory skin disorder affecting the scalp, face, and torso. Typically, seborrheic dermatitis presents with scaly, flaky, itchy, and red skin. It particularly affects the sebaceous-gland-rich areas of skin. In adolescents and adults, seborrheic dermatitis usually presents as scalp scaling similar to dandruff or as mild to marked erythema of the nasolabial fold.

Corns and calluses: Corns and calluses develop in areas of skin exposed to repeated friction or pressure. In response, thick layers of dead skin cells pile up and harden.

Warts: Warts are small bumps on the skin that are caused by human papilloma virus (HPV) infection. Plantar warts grow on the soles of the feet.

HPV: Human papilloma virus usually is spread by direct contact. It is typically spread by touching or shaking hands with someone who already has a wart. It may also be spread by coming in contact with a contaminated surface. For example, by walking barefoot on a gym floor or a pool deck or by wearing someone else's shoes.

Chronic eczema: Eczema, also known as dermatitis, is an inflammation of the skin. It can be triggered by allergies, irritating chemicals and other factors.

Lichen planus: This condition may appear as a lacy white patch on the inside of the mouth. Or it may be an itchy, violet, scaly patch elsewhere on the skin. Lichen planus may be related to an abnormal reaction of the immune system.

Actinic keratosis: These are flat, red, rough, sandpaper-like spots or patches of skin. They can be as tiny as a few millimeters. They are caused by excessive exposure to the ultraviolet radiation of sunlight. They occur on sun-exposed areas of skin. And they have the potential to develop into skin cancer.

Seborrheic keratosis: These are small, noncancerous skin growths. They can be tan, brown or black. They appear on the face, trunk, arms or legs. Seborrheic hyperkeratoses are very common.

Inherited conditions: Several inherited conditions such as ichthyosis cause hyperkeratosis. They cause a widespread, thick, platelike scaling of the skin. Symptoms begin either shortly after birth or during early childhood.

Treatment with keratolytic agents dissolves the inter-cellular matrix or the intracellular desmosomes, promoting desquamation of scaly skin, and eventually results in softening of such abnormal keratotic areas. Keratostatic agents inhibit keratinocytes cells turnover. Keratolytic agents are believed to function by relaxing the cohesiveness of the stratum corneum (SC), this involves the disintegration of desmosomes and hemidesmosomes, which link keratinocytes and bind them to the extracellular matrix (ECM), respectively. Another form of keratolytic activity interferes with the binding of sulfide to sulfide (S—S bonds which provide strength to the keratin filament). Salicylic acid is an example of a keratolytic agent working through the disintegration of desmosomes and hemidesmosomes and selenium disulfide has been implicated as interacting to weaken the disulfide bonds. Other such agents known to be keratolytic and in common use are salicylic acid, sulfur, zinc pyrithione, tar, boric acid, urea, benzoyl peroxide and retinoic acid.

### Cosmetic Uses of Keratolytic Agents

Keratolytic agents are widely used in cosmetics, both in hair products such as shampoos and conditioners, and in skin care products such as creams, ointments and suspensions. Keratolytics possess keratin softening properties and help in exfoliating excess of the horny layer, resulting in a smooth and improved skin appearance. One of the most effective and commonly used keratolytic agents in cosmetology is urea.

### Meibomian Gland

The meibomian glands are large sebaceous glands located in the eyelids and, unlike skin, are unassociated with hair. The meibomian glands produce the lipid layer of the tear film that protects it against evaporation of the aqueous phase. The meibomian gland orifice is located on the epithelial side of the lid margin, and is only a few hundred microns from the mucosal side. The glands are located on both upper and lower eyelids, with higher amounts of the glands on the upper eyelid. A single meibomian gland is composed of clusters of secretory acini that are arranged circularly around a long central duct and connected to it by short ductules. The terminal part of the central duct is lined by an ingrowth of the epidermis that covers the free lid margin and forms a short excretory duct that opens as an orifice at the posterior part of the lid margin just anterior to the mucocutaneous junction near the inner lid border. The oily secretion composed of lipids is produced within the secretory acini. The lipid secretion is a liquid at near body temperature and is delivered to the skin of the lid margin as a clear fluid, called “meibum.” It forms shallow reservoirs on the upper and lower lid margins, and consists of a complex mixture of cholesterol, wax, cholesteryl esters, phospholipids, with small amounts of triglycerides, triacylglycerols, and hydrocarbons. The separate meibomian glands are arranged in parallel, and in a single row throughout the length of the tarsal plates in the upper and lower lids. The term “keratinized obstruction” as used herein refers to a blockage of the meibomian gland, regardless of the location of the blockage. In some embodiments, the blockage is complete, whereas in other embodiments, the blockage is partial. Regardless of the degree of blockage, such keratinized obstruction leads to meibomian gland dysfunction. In some embodiments, the keratinized obstruction is composed of keratinized material and lipids. In some embodiments, the keratinized obstruction is a blockage at the meibomian gland orifice and excretory duct. In some embodiments, the keratinized obstruction is caused by keratinization of the epithelium at the lid margin and meibomian gland. In certain instances, the keratin obstruction is influenced by the migration or aberrant differentiation of stem cells. In some embodiments, the keratinized obstruction results in reduced delivery of oil to the lid margin and tear film, and stasis inside the meibomian gland that causes increased pressure, resultant dilation, acinar atrophy, and low secretion. In certain instances, keratinization of the meibomian gland causes degenerative gland dilation and atrophy.

### Meibomian Gland Dysfunction (MGD)

The term, “meibomian gland dysfunction,” as used herein, refers to chronic, diffuse abnormality of the meibomian glands, that is characterized by terminal duct obstruction or qualitative or quantitative changes in the glandular secretion, or both. MGD may result in alteration of the tear film, eye irritation symptoms, inflammation, or ocular surface disease. The most prominent aspects of MGD are obstruction of the meibomian gland orifices and terminal ducts and changes in the meibomian gland secretions.

MGD is a leading contributor of dry eye syndrome. The occurrence of dry eye syndrome is widespread and affects about 20 million patients in the United States alone. Dry eye syndrome is a disorder of the ocular surface resulting from either inadequate tear production or excessive evaporation of moisture from the surface of the eye. Tears are important to corneal health because the cornea does not contain blood vessels, and relies on tears to supply oxygen and nutrients. Tears and the tear film are composed of lipids, water, and mucus, and disruption of any of these can cause dry eye. An inadequate amount of lipids flowing from the meibomian glands as caused by a keratinized obstruction, may cause excessive evaporation, thereby causing dry eye syndrome.

MGD is not synonymous with posterior blepharitis, which describes inflammatory conditions of the posterior lid margin. MGD may contribute to posterior blepharitis, but MGD may not always be associated with inflammation or posterior blepharitis. MGD also refers to functional abnormalities of the meibomian gland, while “meibomian gland disease,” describes a broad range of meibomian gland disorders, that includes neoplasia and congenital disease. Clinical signs of MGD include meibomian gland dropout, altered meibomian gland secretion, and changes in lid morphology.

In some embodiments, altered meibomian gland secretion is detected by physically expressing the meibomian glands by applying digital pressure to the tarsal plates. In subjects without MGD, the meibum is a pool of clear oil. In MGD, both the quality and expressibility of the expressed material is altered. The altered meibum is also known as meibomian excreta and is made up of a mixture of altered secretions and keratinized epithelial material. In MGD, the quality of expressed lipid varies in appearance from a clear fluid, to a viscous fluid containing particulate matter and densely opaque, toothpaste-like material. The meibomian orifices may exhibit elevations above surface level of the lid, which is referred to as plugging or pouting, and is due to obstruction of the terminal ducts and extrusion of a mixture of meibomian lipid and keratinized material.

Obstructive MGD is characterized by all or some of the following: 1) chronic ocular discomfort, 2) anatomic abnormalities around the meibomian gland orifice (which is one or more of the following: vascular engorgement, anterior or posterior displacement of the mucocutaneous junction, irregularity of the lid margin) and 3) obstruction of the meibomian glands (obstructive findings of the gland orifices by slit lamp biomicroscopy (pouting, plugging or ridge), or decreased meibum expression by moderate digital pressure).

Meibomian gland expressibility is optionally determined to assess the meibomian gland function. In normal patients, meibum is a clear to light yellow oil. Meibum is excreted from the glands when digital pressure is placed on the glands. Changes in meibomian gland expressibility are one potential indicator of MGD. In some embodiments, during expression, quantifying the amount of physical force applied during expression is monitored in addition to assessing lipid volume and lipid quantity.

Current treatments for MGD include lid warming, lid massage, lid hygiene, lid expression and meibomian gland probing. Pharmacological methods, prior to those described herein, have not been used.

Lid hygiene is considered the primary treatment for MGD and consists of three components:

1) application of heat, 2) mechanical massage of eyelids and 3) cleansing the eyelid. Eyelid warming procedures improve meibomian gland secretion by melting the pathologically altered meibomian lipids. Warming is achieved by warm compresses or devices. Mechanical lid hygiene includes the use of scrubs, mechanical expression and cleansing with various solutions of the eyelashes and lid margins. Lid margins are optionally also cleansed with hypoallergenic bar soap, dilute infant shampoo or commercial lid scrubs. Physical expression of meibomian glands is performed in a physician's office or is performed by the patient at home. The technique varies from gentle massage of the lids against the eyeball to forceful squeezing of the lids either against each other or between a rigid object on the inner lid surface and a finger, thumb, or rigid object (such as a glass rod, Q-tip, or metal paddle) on the outer lid surface. The rigid object on the inner lid surface protects the eyeball from forces transferred through the eyelid during expression and to offer a stable resistance, to increase the amount of force that is applied to the glands.

Eyelid warming is limited because the warming melts the lipids, but does not address movement of the keratinized material. Further, eyelid warming induces transient visual degradation due to corneal distortion. Mechanical lid hygiene is also limited because the force needed to remove an obstruction can be significant, resulting in significant pain to the patient. The effectiveness of mechanical lid hygiene is limited by the patient's ability to tolerate the associated pain during the procedure. Other treatments for MGD are limited.

Physical opening of meibomian glands obstruction by meibomian gland expression is an acceptable method to improve meibomian gland secretion and dry eye symptoms. In addition probing of the meibomian gland canal has been used to open the obstructed canal. Both methods, expression and probing, are limited, however, by the pain induced by the procedure, the possible physical insult to the gland and canal structures and their short lived effect estimated at days and weeks. Therefore, methods are needed to improve patient comfort, which will not cause harm to the meibomian glands and canals, that will reduce the dependency on frequent office visits and improve secretion of meibum.

In summary, each of these treatments has a different shortcoming and the treatment of MGD remains challenging. Therefore, methods are needed to improve patient comfort, which will not cause harm to the meibomian glands and canals, that will reduce the dependency on frequent office visits and improve secretion of meibum.

Keratolytic and/or Keratoplastic Agent

Keratolytic agents are widely used in the fields of dermatology for both skin disorders and as cosmetic products due to their property of loosening or removing/exfoliating the horny outer layer of the skin, resulting in keratin softening. Keratolytics are very useful in treating skin conditions involve keratinization. They are used to treat dandruff, psoriasis, acne, warts, corns, calluses, palmoplantar keratodermas, ichthyosis, seborrheic dermatitis and other forms of hyperkeratosis disorders.

In hyperkeratotic disorders, keratinocyte deviations in proliferation, adhesion, and differentiation obstruct the infundibulum and the sebaceous duct, paving the way for excessive sebum secretion, bacterial overgrowth, and inflammatory response due to release of bacterial and cellular products, i.e. sebum and keratin. These conditions are many times accompanied by dermatitis, with irritated, erythematous secondary infected skin.

Hyperkeratosis may be diffused, covering areas of skin or hair (like in the sole of the foot), or follicular, where the excessive development of keratin accumulated in hair follicles or gland orifices resulting in obstruction, cone-shaped, elevated capping. The openings are often closed with a mixture of keratin and sebum (like in acne) or a mixture of keratin and meibum (like in meibomian gland dysfunction)

In some embodiments the keratolytic and/or keratoplastic agent is a selenium-containing amino acid. In some embodiments the selenium-containing amino acid is selenium methionine. In some embodiments the selenium-containing amino acid is selenium cysteine. Selenium methionine (selenomethionine or SeMet) is a water-soluble selenium-containing amino acid. Selenium methionine is primarily used as a food supplement, since selenium is available and absorbable. Selenium methionine metabolism is closely linked to protein turnover. At a constant rate of intake in the nutritional range, tissue Se levels increase until a steady state is established, preventing the build-up to toxic levels. Similarly, the release of selenium methionine from body proteins by catabolic processes during an illness should not result in selenium toxicity because no mechanism for the selective release of selenium methionine during catabolism exists. (Schrauzer 2000. J. Nutr., Vol. 130, No. 7, p. 1653-1656).

In some embodiments, the selenium-containing amino acid has a keratolytic effect on human keratocytes in-vitro (by thiol degradation), as well as keratostatic effect (by inhibition of keratocytes proliferation).

Described herein are compositions comprising a selenium-containing amino acid as a keratolytic agent in a topical cosmetic and/or dermatological formulation and/or ophthalmic formulation, in particular as an active agent to treat conditions which involves abnormal keratinization.

In some embodiments the keratolytic and/or keratoplastic agent is a selenium-containing amino acid selected from the group consisting of:

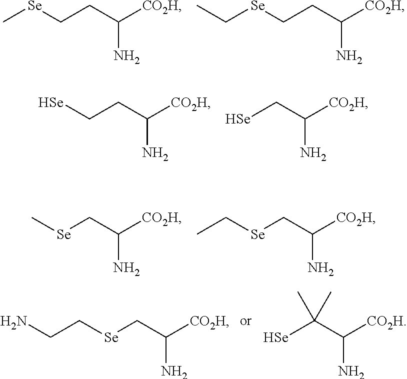

In some embodiments the keratolytic and/or keratoplastic agent is a selenium-containing amino acid selected from the group consisting of:

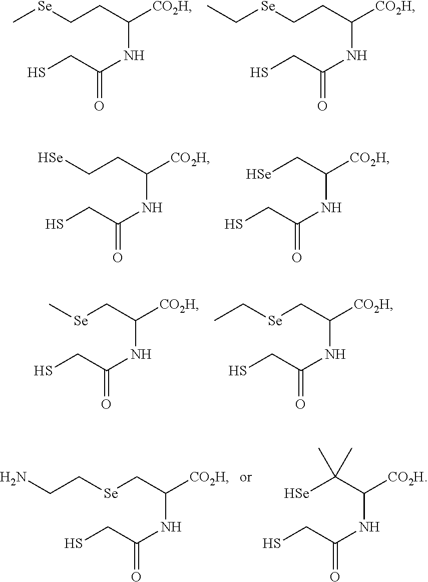

In some embodiments the keratolytic and/or keratoplastic agent is a selenium-containing amino acid selected from the group consisting of:

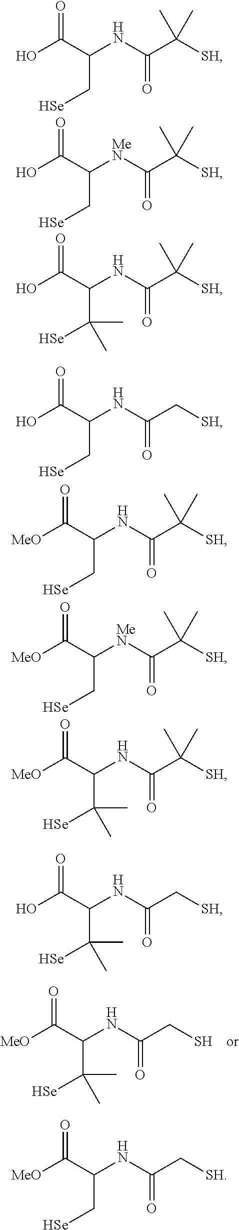

In some embodiments the keratolytic and/or keratoplastic agent is a selenium-containing amino acid selected from the group consisting of:

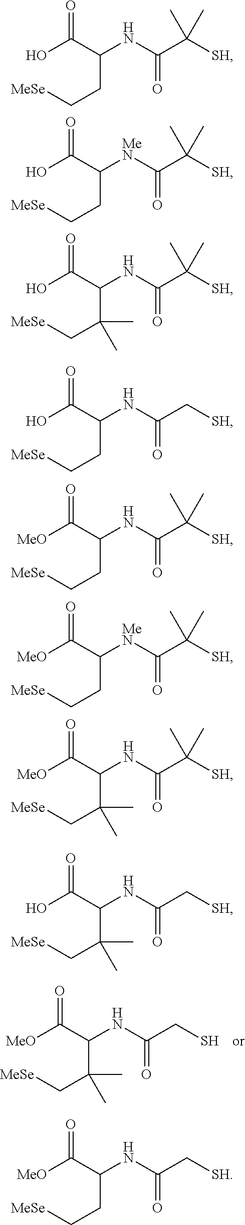

In some embodiments, the composition further comprises an additional keratolytic or keratostatic agent with additive or synergistic pharmacological effect selected from benzoyl peroxide, coal tar, dithranol, salicylic acid, retinoic acid, alpha-hydroxy acid, urea, lactic acid, and selenium disulfide.

In some embodiments, the selenium-containing amino acid has both keratolytic (keratin softening) and keratostatic effect (inhibition of cell cycle at the S-phase). In some embodiments the selenium-containing amino acid is selenium methionine.

In some embodiments, the selenium-containing amino acid has a different mechanism of action than the most common keratolytic agent (for example salicylic acid and selenium disulfide). In some embodiments, the selenium-containing amino acid and the common keratolytic agent share keratolytic effect but are different in the presence of the keratostatic effect (effect on cell cycle). In some embodiments, the selenium-containing amino acid affects the S-phase of the cell cycle. In some embodiments, the selenium-containing amino acid does not affect the Go-phase of the cell cycle.

In some embodiments, the selenium-containing amino acid, affecting the S-phase of the cell cycle, is combined with another keratolytic agent. In some embodiments, the combination has a synergetic effect.

The keratolytic and keratoplastic agents described herein are useful either as an acute therapy (e.g., by a trained specialist or physician) or as a chronic therapy (e.g., in the hands of a patient, or alternatively, by a trained specialist or physician). The agents are tested, in certain embodiments, using the assays and methods described herein (e.g., as described in the examples).

In certain embodiments, mild or weak keratolytic and/or keratoplastic agents are used in the methods and formulations described herein, e.g., with subjects that produce low levels of keratin. Such mild or weak keratolytic and/or keratoplastic agents are optionally used in a maintenance therapy setting. Mild or weak keratolytic and/or keratoplastic agents include lower concentrations of active keratolytic and/or keratoplastic agents, as well as keratolytic and/or kerotoplastic agents that have low inherent activity (as determined, e.g., by the methods described herein). In certain embodiments, the mild or weak keratolytic and/or keratoplastic agent is a selenium-containing amino acid.

In some embodiments, the formulation comprising the keratolytic and/or keratoplastic agent further includes an additional therapeutic agent that is not a meibomian gland opening pharmacological agent. In some embodiments the formulation does not contain jojoba wax or jojoba extract. In some embodiments the formulation does not include boric acid. In some embodiments, the formulation does not include retinoic acid. Alternatively, in some embodiments, the formulation with the keratolytic and/or keratoplastic agent excludes any additional therapeutic agent, other than an optional additional meibomian gland opening pharmacological agent.

One embodiment provides a composition for treating hyperkeratosis disorders in a patient in need thereof comprising a selenium-containing amino acid as a keratolytic agent and wherein the composition is suitable for topical administration to the skin in a pharmaceutical or cosmetic dosage form. Another embodiment provides the composition wherein the selenium-containing amino acid is selenium methionine, or a pharmaceutically acceptable salt thereof. Another embodiment provides the composition wherein the selenium-containing amino acid is selenium cysteine, or a pharmaceutically acceptable salt thereof.

Another embodiment provides the composition wherein the composition comprises the selenium-containing amino acid in a liquid solution formulation for ophthalmic, dermatological, or cosmetic use. Another embodiment provides the composition wherein the composition comprises the selenium-containing amino acid in a suspension for ophthalmic, dermatological, or cosmetic use. Another embodiment provides the composition wherein the composition is a liquid gel for ophthalmic, dermatological, or cosmetic use.

Another embodiment provides the composition wherein the composition is a cream for ophthalmic, dermatological, or cosmetic use. Another embodiment provides the composition wherein the composition is an emulsion for ophthalmic, dermatological, or cosmetic use. Another embodiment provides the composition wherein the composition is a lotion for ophthalmic, dermatological, or cosmetic use. Another embodiment provides the composition wherein the composition is an ointment for ophthalmic, dermatological, or cosmetic use.

One embodiment provides a composition for treating hyperkeratosis disorders in a patient in need thereof comprising a selenium-containing amino acid as a keratolytic agent and wherein the composition is suitable for topical administration to the skin in a pharmaceutical or cosmetic dosage form, wherein the composition further comprises an additional keratolytic or keratostatic agent selected from benzoyl peroxide, coal tar, dithranol, salicylic acid, retinoic acid, alpha-hydroxy acid, urea, lactic acid and selenium disulfide.

One embodiment provides a composition for treating hyperkeratosis disorders in a patient in need thereof comprising a selenium-containing amino acid as a keratolytic agent and wherein the composition is suitable for topical administration to the skin in a pharmaceutical dosage form, wherein the composition is administered via a depot formulation and the depot further comprises a pressure sensitive adhesive, wherein the pressure sensitive adhesive is a rubber based pressure sensitive adhesive, a silicone based pressure sensitive adhesive, or an acrylic based pressure sensitive adhesive.

### Methods of Treatment

One embodiment provides a method for treating a hyperkeratosis disorder in a patient in need thereof comprising topically administering to the skin or eyelid margin of the patient a composition comprising selenium methionine or selenium cysteine formulated as an ophthalmic, dermatological, or cosmetic dosage form.

Another embodiment provides the method wherein the composition is formulated as a solution for ophthalmic, dermatological, or cosmetic use. Another embodiment provides the method wherein the composition is formulated as a suspension for ophthalmic, dermatological, or cosmetic use. Another embodiment provides the method wherein the composition is formulated as a lotion for ophthalmic, dermatological, or cosmetic use. Another embodiment provides the method wherein the composition is formulated as a cream for ophthalmic, dermatological, or cosmetic use. Another embodiment provides the method wherein the composition is formulated as an ointment for ophthalmic, dermatological, or cosmetic use. Another embodiment provides the method wherein the composition is formulated as a gel for ophthalmic, dermatological, or cosmetic use. Another embodiment provides the method wherein the composition is formulated as an emulsion for ophthalmic, dermatological, or cosmetic use

One embodiment provides a method for treating a hyperkeratosis disorder in a patient in need thereof comprising topically administering to the skin or eyelid margin of the patient a composition comprising selenium methionine or selenium cysteine formulated as an ophthalmic, dermatological, or cosmetic dosage form, wherein the composition for topical administration further comprises an additional keratolytic or keratostatic agent selected from benzoyl peroxide, coal tar, dithranol, salicylic acid, retinoic acid, alpha-hydroxy acid, urea, lactic acid and selenium disulfide.

One embodiment provides a method for treating a hyperkeratosis disorder in a patient in need thereof comprising topically administering to the skin or eyelid margin of the patient a composition comprising selenium methionine or selenium cysteine formulated as an ophthalmic, dermatological, or cosmetic dosage form, wherein the method comprises administration from a depot formulation and the depot formulation further comprises a pressure sensitive adhesive, wherein the pressure sensitive adhesive is a rubber based pressure sensitive adhesive, a silicone based pressure sensitive adhesive, or an acrylic based pressure sensitive adhesive.

One embodiment provides a method for treating a hyperkeratosis disorder in a patient in need thereof comprising topically administering to the skin or eyelid margin of the patient a composition comprising selenium methionine or selenium cysteine formulated as an ophthalmic, dermatological, or cosmetic dosage form, wherein the hyperkeratosis disorder is selected from dandruff, psoriasis, acne vulgaris, warts, corns, calluses, palmoplantar keratodermas, ichthyosis, sseborrheic dermatitis, meibomian gland dysfunction, HPV infection, lichen planus, actinic keratosis, and seborrheic keratosis. Another embodiment provides the method wherein the hyperkeratosis disorder is meibomian gland dysfunction.

Disclosed herein are methods for treating hyperkeratosis disorders such as seborrheic dermatitis, psoriasis, ichthyosis, acne vulgaris, meibomian gland dysfunction, anti-dandruff, corns, calluses, warts, HPV infection, lichen planus, actinic keratosis, seborrheic keratosis, etc with compositions comprising a selenium-containing amino acid as a keratolytic and/or keratostatic agent wherein the composition is suitable for topical administration to the skin in a pharmaceutical or cosmetic dosage form.

In some embodiments, the methods for treating the hyperkeratosis disorders further comprises an additional keratolytic or keratostatic agent with additive or synergistic pharmacological effect selected from benzoyl peroxide, coal tar, dithranol, salicylic acid, retinoic acid, alpha-hydroxy acid, urea, lactic acid and selenium disulfide.

One embodiment provides a method for treating meibomian gland dysfunction in a patient in need thereof comprising topical administration of a composition comprising a meibomian gland opening pharmacological agent, wherein the meibomian gland opening pharmacological agent is a keratolytic agent or keratoplastic agent. In some embodiments, the meibomian gland opening pharmacological agent is a selenium-containing amino acid. In some embodiments the keratolytic agent is selenium methionine. In some embodiments the keratolytic agent is selenium cysteine.

One embodiment provides a method for treating meibomian gland dysfunction in a patient in need thereof, comprising topically administering to the patient a composition that reaches the eyelid margin of the patient, wherein the composition comprises a therapeutically-effective amount of at least one keratolytic agent in an ophthalmically-acceptable carrier. In one embodiment, the keratolytic agent is a selenium-containing amino acid. In some embodiments the selenium-containing amino acid is selenium methionine. In some embodiments the selenium-containing amino acid is selenium cysteine. In some embodiments, more than one keratolytic agent is used.

One embodiment provides a method for removing a keratin obstruction of the meibomian gland in a patient having a hyperkeratosis disorder comprising topically administering to the eyelid margin of the patient a composition comprising a therapeutically-effective amount of at least one keratolytic agent in an ophthalmically-acceptable carrier, wherein the hyperkeratosis disorder is selected from meibomian gland dysfunction, or dry eye. Another embodiment provides the method wherein the hyperkeratosis disorder is meibomian gland dysfunction. Another embodiment provides the method wherein the hyperkeratosis disorder is dry eye. In one embodiment, the keratolytic agent is a selenium-containing amino acid. In some embodiments the selenium-containing amino acid is selenium methionine. In some embodiments the selenium-containing amino acid is selenium cysteine. In some embodiments, more than one keratolytic agent is used.

One embodiment provides a method for removing a keratin obstruction of the meibomian gland in a patient having a blocked, or partially blocked, meibomian gland comprising topically administering to the eyelid margin of the patient a composition comprising a therapeutically-effective amount of at least one keratolytic agent in an ophthalmically-acceptable carrier. In one embodiment, the keratolytic agent is a selenium-containing amino acid. In some embodiments the selenium-containing amino acid is selenium methionine. In some embodiments the selenium-containing amino acid is selenium cysteine. In some embodiments, more than one keratolytic agent is used.

One embodiment provides a method for treating an ophthalmic disorder caused by keratin obstruction of the meibomian gland in a patient in need thereof comprising topically administering to the eyelid margin of the patient a composition comprising a therapeutically-effective amount of at least one keratolytic agent in an ophthalmically-acceptable carrier, wherein the ophthalmic disorder is meibomian gland dysfunction or dry eye. Another embodiment provides the method wherein the ophthalmic disorder is meibomian gland dysfunction. Another embodiment provides the method wherein the ophthalmic disorder is dry eye. In one embodiment, the keratolytic agent is a selenium-containing amino acid. In some embodiments the selenium-containing amino acid is selenium methionine. In some embodiments the selenium-containing amino acid is selenium cysteine. In some embodiments, more than one keratolytic agent is used.

In some embodiments, administration of a keratolytic agent to a keratin obstruction results in proteolysis of desmosomes forming tight junctions between keratinocytes. In some embodiments, administration of a keratolytic agent results in lysis, including the hydrolysis of disulfide bonds. In some embodiments administration of a keratolytic agent reduces the production of keratin.

One embodiment provides a method for treating meibomian gland dysfunction in a patient in need thereof by administering a topical composition comprising a keratolytic agent, wherein the keraloytic agent is a selenium-containing amino acid. In some embodiments, the composition comprises 0.1% to 10% of a selenium-containing amino acid. In some embodiments, the composition comprises at least 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, or greater of a selenium-containing amino acid. In some embodiments, the composition comprising a selenium-containing amino acid is a semi-solid. In some embodiments, the composition comprising a selenium-containing amino acid is a lotion. In some embodiments, the composition comprising a selenium-containing amino acid is a cream. In some embodiments, the composition comprising a selenium-containing amino acid is an ointment. In some embodiments, the composition comprising a selenium-containing amino acid is a suspension. In some embodiments, the composition comprising a selenium-containing amino acid is a solution. In some embodiments the composition containing a selenium-containing amino acid enhances lipid production from the meibomian glands.

In some embodiments, the selenium-containing amino acid composition for topical administration is a liquid, a semi-solid, or a solid. In some embodiments, the composition for topical administration is a solution eye drops or ointment or a solid insert for slow release of the active agent. In some embodiments, the composition for topical administration is an emulsion semi-solid. In some embodiments, the composition for topical administration is a cream. In some embodiments, the composition for topical administration is an ointment. In some embodiments, the composition for topical administration is a lotion. In some embodiments, the composition for topical administration is a gel. In some embodiments, the composition for topical administration is a dispersion. In some embodiments, the composition for topical administration is a suspension.

In some embodiments, the composition for topical administration is an immediate release eye drops or prolonged or sustained release device or ointment or particulate matter.

### Pharmaceutical Excipient

In other embodiments, the compositions described herein are combined with a pharmaceutically suitable or acceptable carrier (e.g., a pharmaceutically suitable (or acceptable) excipient, physiologically suitable (or acceptable) excipient, or physiologically suitable (or acceptable) carrier). Exemplary excipients are described, for example, in Remington: The Science and Practice of Pharmacy (Gennaro, 21 ^{st} Ed. Mack Pub. Co., Easton, Pa. (2005)).

### Certain Definitions

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 10% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

The terms “treat,” “treating,” or “treatment” as used herein, include reducing, alleviating, abating, ameliorating, relieving, or lessening the symptoms associated with the keratosis condition in either a chronic or acute therapeutic scenario.

The term “dispersion” as used herein refers to a system in which particles are dispersed in a continuous phase of a different composition or state. The dispersions are solid dispersions.

The term “lotion” describes an emulsion liquid dosage form. This dosage form is generally for external application to the skin (US FDA Drug Nomenclature Monograph, number C-DRG-00201).

The term “cream” describes an emulsion semisolid dosage form, usually containing>20% water and volatiles and/or <50% hydrocarbons, waxes or polyols as the vehicle. A cream is more viscous than a lotion. This dosage form is generally for external application to the skin (US FDA Drug Nomenclature Monograph, number C-DRG-00201).

The term “ointment” describes a semisolid dosage form, usually containing<20% water and volatiles and/or >50% hydrocarbons, waxes, or polyols as the vehicle. This dosage form is generally for external application to the skin or mucous membranes (US FDA Drug Nomenclature Monograph, number C-DRG-00201).

The term “solution” describes a clear, homogeneous liquid dosage form that contains one or more chemical substances dissolved in a solvent or mixture of mutually miscible solvents (US FDA Drug Nomenclature Monograph, number C-DRG-00201).

The term “suspension” refers to a heterogeneous mixture containing solid particles that are sufficiently large for sedimentation.

### EXAMPLES

### Section 1-Biological Evaluation

Several examples are described below that demonstrate the keratostatic and/or keratolytic efficacy of selenium-containing amino acids. First, a dose response and time course analyses were performed to evaluate the impact of selenium methionine on keratinocyte cells proliferation and viability. Then, selected concentrations were further evaluated by cell cycle analyses (FACS Flow Cytometry and BrdU incorporation (bromodeoxyuridine is a synthetic nucleoside that is an analog of thymidine commonly used in the detection of proliferating cells in living tissues)) to determine the ability of selenium methionine to reduce their proliferation rate both in vitro and ex vivo. The studies also included positive controls, staurosporine, a known cell cycle arrest agent and negative untreated control. Lastly, the ability of selenium methionine to reduce thiol moieties (keratolytic effect) was tested using a degradation assay on human stratum corneum tissues. Salicylic acid (SA), a known keratolytic agent, was used as another control and was evaluated under the same tests.

### Example 1: Selenium Methionine-Induced Reduction of Keratinocytes Proliferation In Vitro: Dose Response and Time Course Analyses

Assessment of keratinocyte turnover rate by selenium methionine was carried out in a certified HaCaT cell line (an immortal human keratinocyte line) obtained originally from CLS GmbH. These cells are routinely used in dermatological studies.

The aim of this preliminary experiment was to determine the appropriate concentration and application time to be evaluated in the main phase.

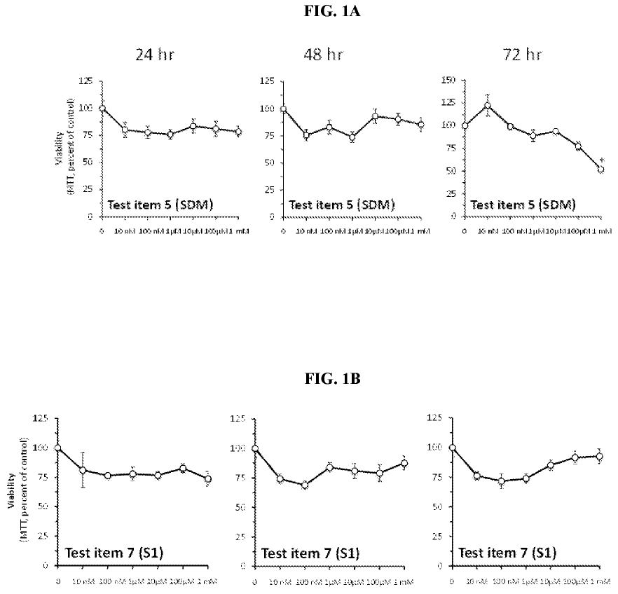

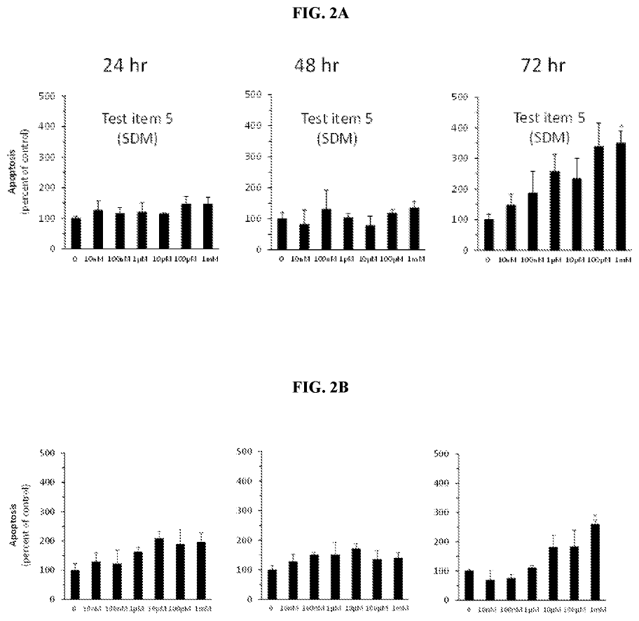

HaCaT cells were seeded in a 96 well plates in concentration of 0.3×10⁶ cells/mL in 2004/well in complete growth medium (DMEM supplemented with 10% FBS and antibiotics). At the start of the experiment (time point 0, 60%-70% optical confluency), the cell viability was recorded by MTT (Example 1, FIG. 1) and Caspase-3 assays (Example 2, FIG. 2). The cells were incubated with or without five concentrations of selenium methionine (10 nM, 100 nM, 1 μM, 10 μM, 10 μM, 1 mM), for 24-, 48- and 72-hr at 37° C. with 5% CO₂ under humidified conditions. Staurosporine (100 nM) and 0.1% SDS served as positive controls in the MTT and Caspase-3 assays, respectively. The assay was carried out in triplicates. Untreated cells were used as negative controls. At the end of each incubation period, cell viability and the extent of apoptosis were measured by MTT and Caspase-3 assays, respectively. A proper blank control was subtracted from the measurements.

FIG. 1A shows that selenium methionine has a significant dose-dependent effect in the reduction of keratinocyte turnover or proliferation rate at 72 hours as determined by the lack of reduction in the MTT cell viability tests.

FIG. 1B shows that SA has no dose dependent effect in the reduction of keratinocyte turnover or proliferation rate at 72 hours as determined by the pronounced reduction in the MTT cell viability tests.

### Example 2: Selenium Methionine-Induced Apoptosis

FIG. 2A shows that selenium methionine has an apoptotic effect in HaCaT keratinocytes cell line at 72 hours as demonstrated by the significant increase in the activation of caspase-3 apoptosis marker. The caspase-3 activation by selenium methionine is in direct correlation to the reduction shown in the MTT cell viability assay in FIG. 1A.

FIG. 2B shows that SA has an apoptotic effect in HaCaT keratinocytes cell line at 72 hours as demonstrated by the significant increase in the activation of caspase-3 apoptosis marker. The caspase-3 activation by SA is with no correlation to the reduction shown in the MTT cell viability assay in FIG. 1B.

### Example 3: Selenium Methionine-Induced Keratostatic Effect (BrdU)

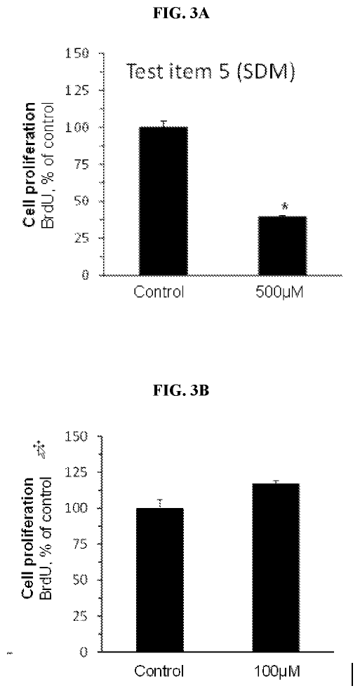

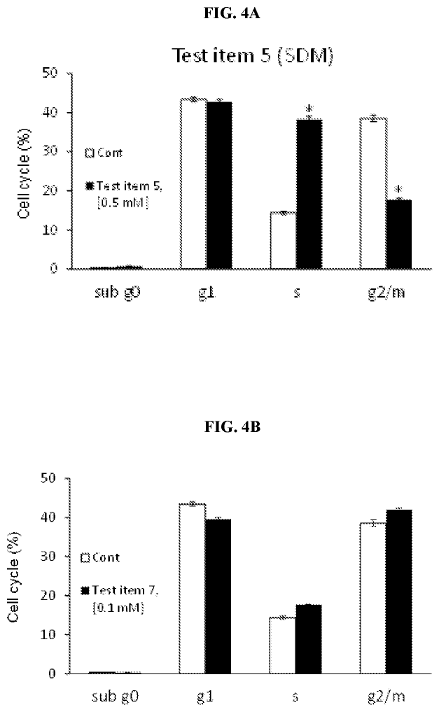

To further investigate the impact of selenium methionine on the cells, the DNA replication rate of the cells was monitored by the BrdU assay. The aim was to determine the ability of selenium methionine at selected concentrations and times to reduce HaCaT keratinocytes cells turnover rate. The assay was carried out in triplicate. The cells were seeded in 6 or 96 well plates for BrdU (FIG. 3) and FACS analysis (Example 4, FIG. 4) respectively, in complete growth medium at 37° C. with 5% CO₂ under humidified atmosphere. The cells were incubated with or without selenium methionine (for the selected time points and concentrations), determined in Example 1. BrdU and FACS analyses were performed in one selected concentration. Two time points (24 hr and 48 hr) were chosen for the flow cytometry and one for the BrdU assay. In order to induce culture synchronization, serum-free medium was used prior of BrdU/FACS analyses in the 24 hr treatment groups.

The BrdU ELISA assay was performed according to kit manufacturer instructions. Briefly, during the final 16 hours of culture, BrdU was added to each well. The cells were fixed, permeabilized, and DNA denatured by the addition of buffers. BrdU monoclonal antibody was pipetted into the wells and allowed to bind for one hour. Colorimetric evaluation of the turnover rate was recorded by ELISA reader. The BrdU assay captures a 16 hr window, which gives a wide view on the proliferation rate of the cells.

FIG. 3A shows that selenium methionine reduced the proliferation of the cells (DNA replication) at 48 hr incubation. The results demonstrate a keratostatic dose-dependent effect of selenium methionine with up to 60% reduction of proliferation of keratocytes, and are in agreement with the cell viability results in Example 1. As expected, staurosporine, the positive control agent, showed a dose-dependent reduction in the proliferation rate of the cells.

FIG. 3B shows that SA does not affect the proliferation of the cells (DNA replication) at 48 hr incubation. The results demonstrate that SA has no keratostatic dose-dependent effect and is in agreement with the cell viability results in FIG. 1B. As expected, staurosporine, the positive control agent, showed a dose-dependent reduction in the proliferation rate of the cells.

### Example 4: Selenium Methionine Cell Arrest Properties (FACS)

The effect of selenium methionine on cell cycle was analyzed by flow cytometry (FACS). At the end of the treatment, the cells were harvested by trypsinization and counted. Approximately 1.3×10⁶ cells were transferred to a new tube, centrifuged for 5 min at 1500 rpm (2-8° C.), and suspended in 0.5 ml of ice cold PBS. Then, the cells were fixed by adding 2.5 ml of 70% ethanol (2-8° C.) in a dropwise manner. The cells were stored at 2-8° C. for 48 hr at this stage. After 30 min of incubation, the samples were washed 3×1 mL PBS and 0.5 mL of ribonuclease stock solution (0.2 mg/mL supplemented with 0.1% (v/v) Triton x-100 in PBS) were added to the cell pellet for 1 hr at rt. This step was performed to ensure only DNA, not RNA, was stained. 0.5 mL of propidium iodide (PI, 100 μg/ml) was added to the samples still suspended in the ribonuclease stock solution. The samples were mixed gently and stained for 30 min. Then, all samples were filtered to reduce cell clumping with a cell strainer (70 μm). All samples were taken immediately to flow cytometric analysis of cell cycle in the FACS apparatus on ice.

Selenium methionine showed cell arrest properties inducing an elongation of the S phase in the cell cycle and lowered M phase (FIG. 4A). Selenium methionine showed a unique effect of S delay of the affected phase in the cell cycle, and therefore may have synergistic effects in a combination with other kerato-modifiyng agents. The effect of selenium methionine is of interest as this MOA is not common to cell cycle arrest agents. Salicylic acid showed no cell arrest properties during all cell cycles (FIG. 4B).

### Example 5: Keratolytic Effect of Selenium Methionine Determined Ex Vivo in Human Skin Tissue by The Thiol Degradation Assay

The thiol oxidoreductase properties of selenium methionine were investigated in isolated stratum corneum obtained from normal human skin. Human skin was obtained from a healthy patient undergoing plastic surgery (male, 27 year old, abdominal). The skin was cleaned, washed with PBS, and then the epidermis was peeled. The epidermis was incubated with 100 mL of 0.005% trypsin (in PBS) at 37° C. overnight. The stratum corneum was washed with HPLC grade water and transferred to a clean Eppendorf tube. The water was evaporated by SpeedVac and the stratum corneum was washed once again with ice cold hexane to exclude residuals lipids in the fraction. The solvent was removed by SpeedVac.

The stratum corneum pieces were weighed in order to determine their dry weight. 50 mg of dried stratum corneum was dissolved in 1M NaOH and mixed thoroughly for 1 hr. Stratum corneum was treated with or without the test items for 2 hr. L-acetylcysteine containing free thiol moieties served as positive control. To isolate the free thiol moieties from the mixture, the samples or blank (50 μl) were incubated with equal volume of TCA (trichloroacetic acid) for 5 min. Then, the tubes were centrifuge for 15 min at 10,000 rpm at room temperature. The pellet was evaluated. Ellman's reagent stock solution (3 mM) was prepared in methanol. The solution was diluted by 11×(1 vol. of reagent with 10 vol. of buffer) in 1M Tris buffer and were mixed gently for 1 min and the solution used immediately. The working solution (220 μl) was transfer into a 96 well plate and 50 μl of the samples, standards or blank were added to the appropriate wells. The 96-well plates were incubated for 5 min at RT, mixed, and the optical absorbance was recorded at 412 nm. A standard calibration curve was read simultaneously without the TCA precipitation step. Sebosel, a commercial selenium sulfide formulation, was used as a reference positive control, and showed an increase in the free thiol moieties by 4 folds.

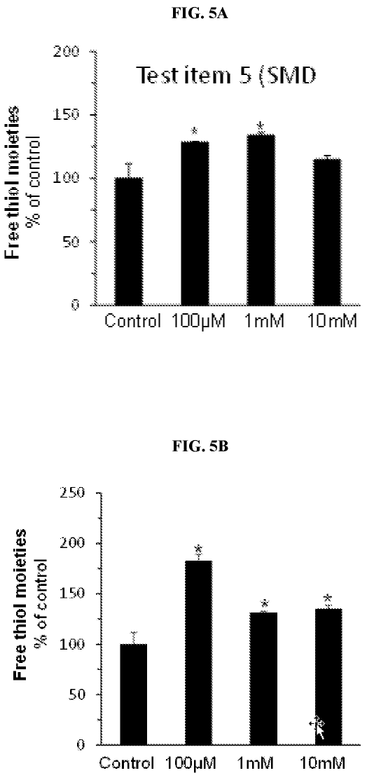

FIG. 5A shows that selenium methionine caused up to 35% elevation in thiol degradation assay releasing free SH moieties and therefore has the therapeutic potential to loosen Keratin.

FIG. 5B shows that SA caused elevation in thiol degradation assay releasing free SH moieties and therefore has the therapeutic potential to loosen keratin.

Keratins have large amounts of the sulfur-containing amino acid cysteine, required for the disulfide bridges that confer strength and rigidity by permanent, thermally stable crosslinking. Thus, it is very difficult to dissolve keratin-containing tissues because of the cross-linked disulfide bridges. These bridges create a helix shape that is extremely strong, as sulfur atoms bond to each other, creating a fibrous matrix that is not readily soluble.

The inorganic and water-insoluble compound selenium disulfide is known to have keratoplastic and keratolytic activity by depression of epidermal cell turnover rate and interference with hydrogen bond formation in the keratin, probably by its ability to inactivate free sulfhydryl groups and compounds through mercaptide formation. The present observed keratolytic effect of the water-soluble selenium-containing amino acid, selenium methionine, is the first reported keratolytic activity of a selenium-containing amino acid.

### Example 6: Selenocysteine-Induced Reduction of Keratinocytes Proliferation In Vitro—Dose Response and Time Course Analyses

Assessment of the effect of selenium cysteine on keratinocyte turnover rate was carried out in a certified HaCaT cell line (an immortal human keratinocyte line) obtained originally from CLS GmbH. These cells are routinely used in dermatological studies. The aim of this preliminary experiment was to determine the appropriate concentration and application time to be evaluated in the main phase.

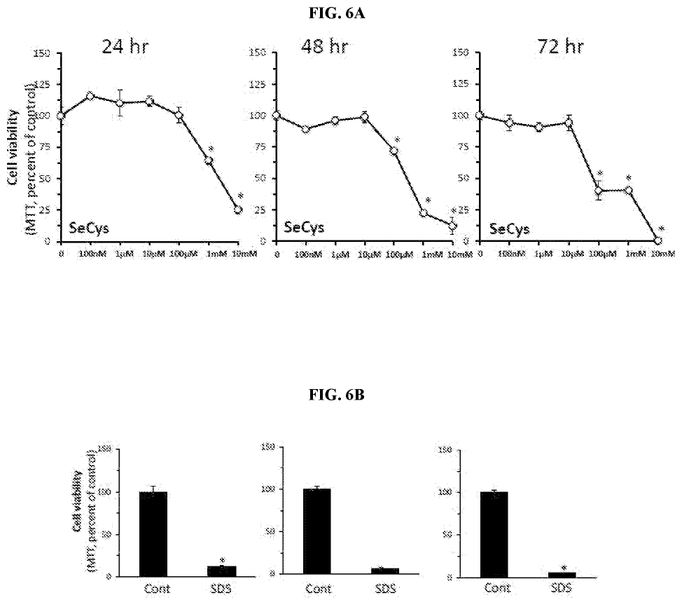

HaCaT cells were seeded in well plates at concentration of 0.3×10⁶ cells/mL in 2004/well in complete growth medium (DMEM supplemented with 10% FBS and antibiotics). At the start of the experiment (time point 0, 60%-70% optical confluency), the cell viability was recorded by MTT. The cells were incubated at five concentrations of selenium cysteine (100 nM, 1 μM, 10 μM, 100 μM, 10 mM), for 24-, 48- and 72-hours at 37° C. with 5% CO₂ under humidified conditions. The assay was carried out in triplicate. Untreated cells were used as negative controls. The positive control was 0.1% SDS (FIG. 6B). At the end of each incubation period, cell viability was measured by MTT.

FIG. 6A shows that selenium cysteine has a significant dose-dependent effect in the reduction of keratinocyte turnover or proliferation rate at 72 hours as determined by the pronounced reduction in the MTT cell viability tests.

### Example 7: Selenocysteine-Induced Keratostatic Effect as Determined by BrdU Incorporation

To further investigate the impact of selenium cysteine on the cells, the DNA replication rate of the cells was monitored by a BrdU incorporation assay. The purpose was to determine the ability of selenium cysteine at selected concentrations and times to reduce HaCaT keratinocytes cells turnover rate. The assay was carried out in triplicates. The positive control was stautosporine (100 nM). The cells were seeded in 6- or 96-well plates in complete growth medium at 37° C. with 5% CO₂ under humidified atmosphere. The cells were incubated with or without selenium cysteine for the selected time points and concentrations determined in Example 6. BrdU incorporation was performed at the selected concentration and a 48 hr time point was chosen for the BrdU assay.

The BrdU ELISA assay was performed according to kit manufacturer instructions. Briefly, during the final 16 hours of culture, BrdU was added to each well. The cells were fixed, permeabilized, and DNA denatured by the addition of buffers. BrdU monoclonal antibody was pipetted into the wells and allowed to bind for one hour. Colorimetric evaluation of the turnover rate was recorded by ELISA reader. The BrdU assay captures a 16 hr window, which gives a wide view on the proliferation rate of the cells.

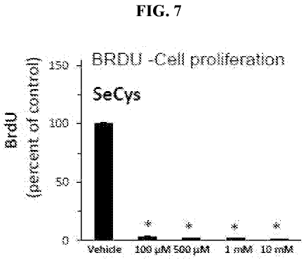

FIG. 7 shows that selenium cysteine reduced the proliferation of the cells as measured by DNA replication at 48 hr incubation. The results demonstrate a keratostatic effect of selenium cysteine with more than 90% reduction of proliferation of keratocytes, and are in agreement with the cell viability results in Example 6.

### Section 2— Pharmaceutical Compositions

### Example 1: Formulation of Selenium Methionine Eye Drops

Provided are formulas for several variations of a formulation of selenium methionine in eye drops. The formulations may further comprise antimicrobial preservatives, anti-oxidants, and/or chelating agents.

| Ingredients | % W/W | % W/W | % W/W | % W/W | % W/W | % W/W |
|---|---|---|---|---|---|---|
| Selenium methionine | 1.0 | 1.0 | 1.0 | 3.0 | 3.0 | 3.0 |
| Hypromelose | 0.5 | | | 0.5 | | |
| Carboxymethyl cellulose | | 0.5 | | | 0.5 | |
| Polyvinyl alcohol | | | 0.5 | | | 0.5 |
| Sodium chloride | 0.6 | 0.6 | 0.6 | 0.6 | 0.6 | 0.6 |
| EDTA | 0.05 | 0.05 | 0.05 | 0.05 | 0.05 | 0.05 |
| Water | To 100 | To 100 | To 100 | To 100 | To 100 | To 100 |

### Example 2: Formulation of Selenium Methionine Eye Gel

Provided are formulas for several variations of a formulation of selenium methionine in an eye gel. The formulations may further comprise antimicrobial preservatives, anti-oxidants, and/or chelating agents.

| Ingredients | % W/W | % W/W | % W/W | % W/W |
|---|---|---|---|---|
| Selenium methionine | 1.0 | 1.0 | 3.0 | 3.0 |
| Carbopol 980 | 1.0 | 1.0 | 1.0 | 1.0 |
| Sodium chloride | 0.6 | 0.6 | 0.6 | 0.6 |
| Boric acid | 0.1 | | 0.1 | |
| Sodium hydrogen | | 0.2 | | 0.2 |
| phosphate | | | | |
| Sodium hydroxide | QS | QS | QS | QS |
| EDTA | 0.05 | 0.05 | 0.05 | 0.05 |
| Water | To 100 | To 100 | To 100 | To 100 |

QS—Sufficient quantity to adjust pH to 6.5 to 7.0

### Example 3: Formulation of Selenium Methionine in Lipophilic Ointment

Provided are formulas for several variations of a formulation of selenium methionine in a lipophilic ointment. The formulations may further comprise antimicrobial preservatives, anti-oxidants, and/or chelating agents.

| Ingredients | % W/W | % W/W | % W/W | % W/W | % W/W | % W/W |
|---|---|---|---|---|---|---|
| Selenium methionine | 1.0 | 1.0 | 3.0 | 3.0 | 5.0 | 5.0 |
| Mineral oil | 10 | 10 | 10 | 10 | — | — |
| Capric/caprylic | 10 | — | 10 | — | 10 | 10 |
| triglyceride | | | | | | |
| Microcrystalline wax | 10 | 20 | 10 | 20 | 10 | 10 |
| Squalane | — | 20 | — | 20 | 10 | 10 |
| Lanolin | — | — | — | — | 5 | 5 |
| Purified water | — | — | — | — | — | 5.0 |
| Vaseline | To 100 | To 100 | To 100 | To 100 | To 100 | To 100 |

### Example 4: Formulation of Selenium Cysteine Eye Drops

Provided are formulas for several variations of a formulation of selenium cysteine in eye drops. The formulations may further comprise antimicrobial preservatives, anti-oxidants, and/or chelating agents.

| Ingredients | % W/W | % W/W | % W/W | % W/W | % W/W | % W/W |
|---|---|---|---|---|---|---|
| Selenium cysteine | 1.0 | 1.0 | 1.0 | 3.0 | 3.0 | 3.0 |
| Hypromelose | 0.5 | | | 0.5 | | |
| Carboxymethyl cellulose | | 0.5 | | | 0.5 | |
| Polyvinyl alcohol | | | 0.5 | | | 0.5 |
| Sodium chloride | 0.6 | 0.6 | 0.6 | 0.6 | 0.6 | 0.6 |
| EDTA | 0.05 | 0.05 | 0.05 | 0.05 | 0.05 | 0.05 |
| Water | To 100 | To 100 | To 100 | To 100 | To 100 | To 100 |

### Example 5: Formulation of Selenium Cysteine Eye Gel

Provided are formulas for several variations of a formulation of selenium cysteine in an eye gel. The formulations may further comprise antimicrobial preservatives, anti-oxidants, and/or chelating agents.

| Ingredients | % W/W | % W/W | % W/W | % W/W |
|---|---|---|---|---|
| Selenium cysteine | 1.0 | 1.0 | 3.0 | 3.0 |
| Carbopol 980 | 1.0 | 1.0 | 1.0 | 1.0 |
| Sodium chloride | 0.6 | 0.6 | 0.6 | 0.6 |
| Boric acid | 0.1 | | 0.1 | |
| Sodium hydrogen | | 0.2 | | 0.2 |
| phosphate | | | | |
| Sodium hydroxide | QS | QS | QS | QS |
| EDTA | 0.05 | 0.05 | 0.05 | 0.05 |
| Water | To 100 | To 100 | To 100 | To 100 |

QS—Sufficient quantity to adjust pH to 6.5 to 7.0

### Example 6: Formulation of Selenium Cysteine in Lipophilic Ointment

Provided are formulas for several variations of a formulation of selenium cysteine in a lipophilic ointment. The formulations may further comprise antimicrobial preservatives, anti-oxidants, and/or chelating agents.

| Ingredients | % W/W | % W/W | % W/W | % W/W | % W/W | % W/W |
|---|---|---|---|---|---|---|
| Selenium cysteine | 1.0 | 1.0 | 3.0 | 3.0 | 5.0 | 5.0 |
| Mineral oil | 10 | 10 | 10 | 10 | — | — |
| Capric/caprylic | 10 | — | 10 | — | 10 | 10 |
| triglyceride | | | | | | |
| Microcrystalline wax | 10 | 20 | 10 | 20 | 10 | 10 |
| Squalane | — | 20 | — | 20 | 10 | 10 |
| Lanolin | — | — | — | — | 5 | 5 |
| Purified water | — | — | — | — | — | 5.0 |
| Vaseline | To 100 | To 100 | To 100 | To 100 | To 100 | To 100 |

1-23. (canceled)

24. An ophthalmic composition for treating a hyperkeratosis disorder in a patient in need thereof, wherein the composition comprises a selenium-containing amino acid as a keratolytic agent and wherein the ophthalmic composition is suitable for topical ophthalmic administration to an eye in a pharmaceutical dosage form.

25. The ophthalmic composition of claim 24, wherein the selenium-containing amino acid is selenium methionine, or a pharmaceutically acceptable salt thereof.

26. The ophthalmic composition of claim 24, wherein the selenium-containing amino acid is selenium cysteine, or a pharmaceutically acceptable salt thereof.

27. The ophthalmic composition of claim 24, wherein the ophthalmic composition is formulated as a liquid solution, liquid gel, suspension, or emulsion.

28. The ophthalmic composition of claim 24, wherein the ophthalmic composition is formulated as a cream, lotion, or ointment.

29. The ophthalmic composition of claim 24, wherein the ophthalmic composition further comprises an additional keratolytic or keratostatic agent selected from the group consisting of benzoyl peroxide, coal tar, dithranol, salicylic acid, retinoic acid, alpha-hydroxy acid, urea, lactic acid, and selenium disulfide.

30. The ophthalmic composition of claim 24, wherein the ophthalmic composition is a depot formulation, wherein the ophthalmic composition further comprises a pressure sensitive adhesive,

31. The ophthalmic composition of claim 30, wherein the pressure sensitive adhesive is a rubber based pressure sensitive adhesive, a silicone based pressure sensitive adhesive, or an acrylic based pressure sensitive adhesive.

32. The ophthalmic composition of claim 24, wherein the hyperkeratosis disorder is meibomian gland dysfunction or dry eye.

33. The ophthalmic composition of claim 24, wherein the ophthalmic composition is suitable for topical ophthalmic administration to an eyelid margin.

34. The ophthalmic composition of claim 24, wherein the selenium-containing amino acid is selenium methionine, or a pharmaceutically acceptable salt thereof.

35. The ophthalmic composition of claim 24, wherein the selenium-containing amino acid is selenium cysteine, or a pharmaceutically acceptable salt thereof.

36. The ophthalmic composition of claim 24, wherein the ophthalmic composition comprises between about 0.1% to about 10% of the selenium-containing amino acid.

37. The ophthalmic composition of claim 24, wherein the ophthalmic composition comprises between about 0.1% to about 10% of selenium methionine.

38. The ophthalmic composition of claim 24, wherein the ophthalmic composition comprises between about 0.1% to about 10% of selenium cysteine.

39. A method for treating a hyperkeratosis disorder in a patient in need thereof comprising topically administering to an eyelid margin of the patient a pharmaceutical formulation comprising selenium methionine or selenium cysteine, or a pharmaceutically acceptable salt thereof.

40. The method of claim 39, wherein the pharmaceutical formulation further comprises a keratolytic or keratostatic agent selected from benzoyl peroxide, coal tar, dithranol, salicylic acid, retinoic acid, alpha-hydroxy acid, urea, lactic acid and selenium disulfide.

41. The method of claim 39, wherein the pharmaceutical formulation is a depot formulation, and wherein the depot further comprises a pressure sensitive adhesive, wherein the pressure sensitive adhesive is a rubber based pressure sensitive adhesive, a silicone based pressure sensitive adhesive, or an acrylic based pressure sensitive adhesive.

42. The method of claim 39, wherein the hyperkeratosis disorder is meibomian gland dysfunction or dry eye.

43. The method of claim 39, wherein the pharmaceutical formulation comprises between about 0.1% to about 10% of selenium methionine or selenium cysteine.
